(19) United States
(12) Patent Application Publication
(10) Pub. No.: US 2013/0072768 A1
(43) Pub. Date: Mar. 21, 2013

(54) INTRAVASCULAR GLUCOSE SENSOR

(71) Applicants: Barry Colin Crane, Shennington (GB); John Gilchrist, Helensburg (GB); Neil Cairns, Paisley (GB)

(72) Inventors: Barry Colin Crane, Shennington (GB); John Gilchrist, Helensburg (GB); Neil Cairns, Paisley (GB)

(21) Appl. No.: 13/579,010

(22) PCT Filed: Feb. 15, 2011
(86) PCT No.: PCT/GB2011/000211

§ 371 Date: Nov. 29, 2012

Related U.S. Application Data
(60) Provisional application No. 61/306,373, filed on Feb. 19, 2010.

(51) Int. Cl. A61B 5/145; A61B 5/1459

(52) U.S. Cl. CPC A61B 5/14532; A61B 5/1459
USPC 600/316

(57) ABSTRACT

A glucose sensor for intravascular measurement of glucose concentration wherein the sensor is arranged to measure glucose concentration by monitoring the lifetime of the fluorophore, the sensor comprising:—an indicator system comprising a receptor for selectively binding to glucose and a fluorophore associated with said receptor, wherein the fluorophore has a life-time of less than 100 ns;—a light source;—an optical fibre arranged to direct light from the light source onto the indicator system; —a detector arranged to receive fluorescent light emitted from the indicator system; and—a signal processor arranged to determine information related to a fluorescence lifetime of the fluorophore based on at least the output signal of the detector.

### FIELD OF THE INVENTION

The present invention relates to a sensor for intravascular measurement of glucose and a method of intravascular glucose measurement.

### BACKGROUND TO THE INVENTION

The treatment of post-surgical patients using “tight glycaemic control” (TCG), i.e. by therapeutic compensation for temporary insulin resistance, has yielded clear improvements in patient outcomes. Similar benefits can be seen by applying this same level of patient care to non-surgical, medical ICU patients and beyond.

Many hospitals have sought to implement TGC via intensive insulin therapy (“IIT”). The greatest deterrents to adopting TGC/IIT are the lack of an appropriate technology to meet customer needs for tight control, ease of use, automated monitoring, and consequent labour implications. Maintaining a patient's glucose level within the target range is difficult using intermittent technologies as this requires frequent measurements to guard against hypoglycaemia and the risk of adverse outcomes. Although already widely adopted, the practice of TGC is problematic for hospitals; currently the monitoring of glucose is performed manually by nursing staff, mainly using finger sticks and glucometers and hence only providing intermittent data with limited accuracy (typically±20% for 95% of the measurements).

To avoid the need for frequent blood sampling, a number of sensors have been developed that measure glucose in interstitial fluid of tissue rather than blood. Such sensors, however, typically show a long physiological response time to glucose when compared to that measured in whole blood. In addition patients that are shocked, particularly those in Intensive Care, very often suffer poor peripheral perfusion and hence changes in whole blood glucose concentrations are not readily transmitted to interstitial fluid.

Non-invasive sensors are under development and would usually be applied to measuring glucose in tissue and therefore suffer from the same disadvantages. The development of non-invasive glucose sensing has also been fraught with significant technical challenges.

Some developers of glucose sensors have taken an ex-vivo approach where blood is sampled from the patient and then flowed over the sensor, placed external to the patient, and then flushed to waste or passed back into the patient. This is at best a rapid intermittent means of measuring glucose and has the disadvantage of cumulatively utilizing significant volumes of patient blood. The maintenance of sterility and blood access lines open is also problematic in such techniques.

The configuration of intravascular optical sensors was defined in the 1980-1990s with the development of multi parameter optical sensors for the intravascular continuous measurement of blood gases, namely, oxygen, carbon dioxide and pH. These equilibrium type receptors for the blood gases were either absorption or fluorescence intensity based indicators. These sensors suffered from drift in their signals over prolonged periods of time and generally required calibration just prior to use. Although the general optical configuration of these blood gas sensors are appropriate for glucose sensing by use of suitable glucose receptor chemistry, there remains a problem with sensor drift and the requirement for calibration.

There is therefore a need for a whole blood glucose sensor, which avoids the difficulties of sensor drift and ideally avoids the need for calibration by the end user.

### SUMMARY OF THE INVENTION

The present invention provides a glucose sensor for intravascular measurement of glucose concentration wherein the sensor is arranged to measure glucose concentration by monitoring the lifetime of the fluorophore, the sensor comprising:
an indicator system comprising a receptor for selectively binding to glucose and a fluorophore associated with said receptor, wherein the fluorophore has a lifetime of less than 100 ns;
a light source;
an optical fibre arranged to direct light from the light source onto the indicator system;
a detector arranged to receive fluorescent light emitted from the indicator system; and
a signal processor arranged to determine information related to a fluorescence lifetime of the fluorophore based on at least the output signal of the detector.

The sensors of the invention accordingly determine the glucose concentration in the blood stream by determining changes in the fluorescence lifetime of the fluorophore.

The fluorescent lifetime of an indicator is an intrinsic property and is independent of changes in light source intensity, detector sensitivity, light through put of the optical system (such as an optical fibre), immobilized sensing thickness and indicator concentration. In addition, photo bleaching of the fluorophore, that translates to signal drift when fluorescence intensity is measured, is of much smaller significance when fluorescent lifetimes are measured. This means that in contrast to intensity based measurements, no compensation for these variables is required when fluorescent lifetimes are measured. Thus for the end user of such a device this means that there is no need for calibration or recalibration. Lifetime measurement of glucose therefore has significant benefits over intensity based measurement in terms of sensor performance, calibration and ease of use for the end user.

However, there are considerable barriers currently to the development of practically useful lifetime measuring devices. The instrumentation required for the accurate measurement of fluorescent lifetimes is at present expensive and bulky. The use of long lifetime (>100 ns) fluorescent metal-ligand/boronic acid complexes as indicators for the optical measurement of glucose can facilitate the use of small, low cost instrumentation, such as a light emitting diode for excitation, a photodiode detector, phase fluorimetry and a look up table. There is a problem, however, in using such long lifetime fluorophores for measuring glucose. Long lifetime fluorophores invariably undergo collisional fluorescence quenching with oxygen and the extent of the quenching is proportional to the unquenched lifetimes. Metal ligand complexes with long fluorescent lifetimes are commonly used for the detection and determination of oxygen. Thus oxygen can be regarded as an intereferent when these long lifetime indicators are used for monitoring glucose in tissue, interstitial fluid or blood or some other body fluid.

The present invention, however, addresses these issues by providing a sensor capable of measuring lifetimes of less than 100 ns using small, low cost instrumentation. The present invention thus enables the benefits of lifetime measurement to be achieved in a device which is suitable for use by a clinician in a hospital environment and which eliminates or reduces the difficulties of oxygen sensitivity.

According to a preferred embodiment, the detector is a single photon avalanche photodiode. In one aspect of this embodiment, the intensity of light emitted by the light source is modulated at a first frequency, and the bias voltage applied to the single photon avalanche photodiode is modulated at a second frequency, different from the first frequency. The bias voltage is above the breakdown voltage of the single photon avalanche photodiode. This selection of bias voltage means that the single photon sensitivity of the detector is maintained, but also has the advantage that a heterodyne measurement approach can be used. In other words, the resulting measurement signal of interest from the single photon avalanche photodiode is at a frequency corresponding to the difference between the first and second frequencies. The first and second frequencies may be of the order of 1 MHz or much higher, but may be selected such that their difference is, for example, of the order of 10 s of kHz.

Therefore, the operational bandwidth of the measurement electronics can be much lower than the first and second modulation frequencies, allowing a simpler design and with less sensitivity to noise.

A further advantageous aspect is to introduce a series of additional phase angles (phase shifts) in the modulation signal for the light source. A series of measurements can then be obtained relating the modulation depth of the measurement signal to the introduced phase angle. Analysing these results can improve the overall precision of the luminescence lifetime measurement.

Also provided by the invention is a method of intravascular measurement of glucose concentration comprising
inserting the indicator system of a sensor of the invention into a vein or artery;
passing incident light from the light source to the indicator system via the optical fibre;
receiving fluorescent light, emitted from the indicator system in response to the light incident on the indicator system from the light source, using the detector and generating an output signal; and
determining information related to the fluorescence lifetime of the fluorophore based on at least the output signal of the detector.

### BRIEF DESCRIPTION OF THE FIGURES

FIGS. 1 and 1a depict a sensor according to the invention.

FIG. 2 schematically depicts a preferred embodiment of the invention.

FIG. 3 is a flowchart of a glucose concentration measurement method according to a preferred embodiment of the invention.

### DETAILED DESCRIPTION OF THE INVENTION

As used herein the term alkyl or alkylene is a linear or branched alkyl group or moiety. An alkylene moiety may, for example, contain from 1 to 15 carbon atoms such as a C₁₋₁₂ alkylene moiety, C₁₋₆ alkylene moiety or a C₁₋₄ alkylene moiety, e.g. methylene, ethylene, n-propylene, i-propylene, n-butylene, i-butylene and t-butylene. C₁₋₄ alkyl is typically methyl, ethyl, n-propyl, i-propyl, n-butyl or t-butyl. For the avoidance of doubt, where two alkyl groups or alkylene moieties are present, the alkyl groups or alkylene moieties may be the same or different.

An alkyl group or alkylene moiety may be unsubstituted or substituted, for example it may carry one, two or three substituents selected from halogen, hydroxyl, amine, (C₁₋₄ alkyl) amine, di(C₁₋₄ alkyl) amine and C₁₋₄ alkoxy. Preferably an alkyl group or alkylene moiety is unsubstituted.

As used herein the term aryl or arylene refers to C₆₋₁₄ aryl groups or moieties which may be mono-or polycyclic, such as phenyl, naphthyl and fluorenyl, preferably phenyl. An aryl group may be unsubstituted or substituted at any position. Typically, it carries 0, 1, 2 or 3 substituents. Preferred substituents on an aryl group include halogen, C₁₋₁₅ alkyl, C₂₋₁₅ alkenyl, —C(O)R wherein R is hydrogen or C₁₋₁₅ alkyl, —CO₂R wherein R is hydrogen or C₁₋₁₅ alkyl, hydroxy, C₁₋₁₅ alkoxy, and wherein the substituents are themselves unsubstituted.

As used herein, a heteroaryl group is typically a 5- to 14-membered aromatic ring, such as a 5- to 10-membered ring, more preferably a 5- or 6-membered ring, containing at least one heteroatom, for example 1, 2 or 3 heteroatoms, selected from O, S and N. Examples include thiophenyl, furanyl, pyrrolyl and pyridyl. A heteroaryl group may be unsubstituted or substituted at any position. Unless otherwise stated, it carries 0, 1, 2 or 3 substituents. Preferred substituents on a heteroaryl group include those listed above in relation to aryl groups.

The present invention provides a sensor and measurement technique for the intravascular measurement of glucose concentration. The sensors of the invention are based on an optical fibre which is arranged to direct light onto an indicator system. The indicator system is provided within a sensing region, which is typically contained in a cell within, or attached to, the distal end of the optical fibre. In use, the distal end of the fibre is inserted into a blood vessel so that the indicator system is located within the blood flow. Glucose is able to enter the sensing region and therefore quickly contacts the indicator system.

On contact of the glucose with the indicator system, binding occurs between the receptor and glucose molecules. The presence of a glucose molecule bound to the receptor causes a change in the fluorescence lifetime of the indicator system. Thus, monitoring of the lifetime of the fluorophore in the indicator system provides an indication of the amount of glucose which is bound to the receptor. The measurement of glucose concentration by monitoring the lifetime decay has previously been described by Lakowicz in Analytical Biochemistry 294, 154-160 (2001). Measurement by phase modulation is described therein but both phase modulation and single photon counting techniques are appropriate for use with the present invention. Phase modulation is preferred.

The indicator system contains at least a receptor that selectively binds to glucose and a fluorophore associated with the receptor. The lifetime of the fluorescence decay of the fluorophore is altered when glucose is bound to the receptor, allowing detection of glucose by monitoring the lifetime of the fluorophore. In one embodiment, the receptor and fluorophore are covalently bound to one another.

Suitable receptors for glucose are compounds containing one or more, preferably two, boronic acid groups. In a particular embodiment, the receptor is a group of formula (I)

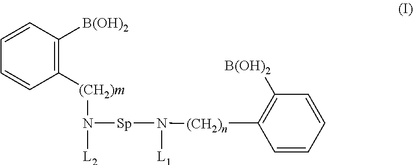

wherein m and n are the same or different and are typically one or two, preferably one; Sp is an alphatic spacer, typically an alkylene moiety, for example a C1-C12 alkylene moiety, e.g. a C6 alkylene moiety; and L1 and L2 represent possible points of attachment to other moieties, for example to a fluorophore. For example, L1 and L2 may represent an alkylene, alkylene-arylene or alkylene-arylene-alkylene moiety, linked to a functional group. Where no attachment to another moiety is envisaged, the functional group is protected or replaced by a hydrogen atom. Typical alkylene groups for L1 and L2 are C1-C4 alkylene groups, e.g. methylene and ethylene, especially methylene. Typical arylene groups are phenylene groups. The functional group is typically any group which can react to form a bond with, for example, the fluorophore or a hydrogel, e.g. ester, amide, aldehyde or azide: In the indicator system, the receptor is typically linked via one or more of these functional groups to the fluorophore and optionally to a support structure such as a hydrogel.

Varying the length of the spacer Sp alters the selectivity of the receptor. Typically, a C6-alkylene chain provides a receptor which has good selectivity for glucose.

Further details of such receptors are found in U.S. Pat. No. 6,387,672, the contents of which are incorporated herein by reference in their entirety. Receptors of formulae (I) and (II) can be prepared by known techniques and details of their synthesis can be found in U.S. Pat. No. 6,387,672.

It is to be understood that the present invention is not limited to the particular receptors described above and other receptors, particularly those having two boronic acid groups, may also be used in the present invention.

Examples of suitable fluorophores include anthracene, pyrene and derivatives thereof, for example the derivatives described in GB 0906318.1, the contents of which are incorporated herein by reference in their entirety. The fluorophore is typically non-metallic. Typically the fluorophore is non-endogenous. The lifetime of the fluorophore is typically 100 ns or less, for example 30 ns or less. The lifetime may be 1 ns or more, for example 10 ns or more, e.g. 20 ns or more. Particular examples of suitable fluorophores are derivatives of anthracene and pyrene with typical lifetimes of 1 to 10 ns and derivatives of acridones and quinacridones with typical lifetimes of 10 to 30 ns.

The receptor and fluorophore are typically bound to one another to form a receptor-fluorophore construct, for example as described in U.S. Pat. No. 6,387,672. This construct may further be bound to a support structure such as a polymeric matrix, or it may be physically entrapped within the probe, for example entrapped within a polymeric matrix or by a glucose-permeable membrane. A hydrogel (a highly hydrophilic cross-linked polymeric matrix such as a cross-linked polyacrylamide) is an example of a suitable polymeric matrix. In a preferred embodiment, a receptor-fluorophore construct is covalently bound to a hydrogel, for example via a functional group on the receptor. Thus, the indicator is in the form of a fluorophore-receptor-hydrogel complex.

In an alternative preferred embodiment, the indicator (i.e. the receptor and fluorophore molecules, or a receptor-fluorophore construct) is provided in aqueous solution, typically the indicator is dissolved in aqueous solution. In this embodiment, the indicator is contained within a cell in the sensor, typically in a cell at or within the distal end of the optical fibre, and a membrane, which is permeable to glucose, provided over any aperture in the cell. In order to ensure that the indicator remains within the cell, it must be of sufficiently high molecular weight to be substantially prevented from leaking out of the cell through the membrane. This can be achieved by selection of a membrane having a suitable molecular weight cut-off, and by providing a high molecular weight indicator.

Providing the indicator (comprising receptor and fluorophore, typically in the from of a receptor-fluorophore construct) as an aqueous solution has the particular advantage to that the microenvironment surrounding each indicator moiety remains substantially constant. Fluorescent sensors can be dramatically influenced by the microenvironment of the indicator. Variation in the localised microenvironment surrounding the indicator can lead to variation in the fluorescent response. In the case of an indicator immobilised onto a polymeric matrix, there is significant variation in the microenvironment, which can lead to a lifetime decay signal in the form of a continuous distribution of decay times and complex multi exponentials. In contrast, where the indicator is dissolved in a water, particularly at low concentrations such that the indicator molecules do not aggregate and are monodispersed, homogeneity is maximum and ideal fluorescent characteristics are achieved for that given solvent. This leads to a signal which is a simple, single exponential.

An alternative means to achieve homogeneity is to immobilise the indicator onto a single molecule support of large molecular weight. Preferably the support is symmetrical and the spatial attachment of the fluorescent indicator is achieved in such a way that the result is also symmetrical. This can, for example, be achieved by the use of a dendrimer as the support material, as discussed below. Thus the environments of each fluorescent indicator molecule attached to such a support will be equivalent. In addition if such a supported molecule can be dissolved in water, at an appropriate concentration, the environments of the supported indicator will be homogenous, again leading to improved signal characteristics.

In this alternative preferred embodiment, therefore, the receptor and fluorophore are bonded to a support material to provide a complex of support, receptor and fluorophore, the complex being dissolved in the solution. The nature of the complex is not important as long as the receptor and fluorophore remain bonded to the support. For example, the support material may be bonded to a receptor-fluorophore construct. Alternatively, the support material may be bonded separately to the fluorophore and to the receptor. In the latter case, the receptor and fluorophore are to not directly bonded to one another but are linked only via the support material. In one embodiment of the invention, the complex takes the form fluorophore-receptor-support.

Typically, a high molecular weight support material is used. This enables the skilled person to restrict the passage of the indicator through the membrane by providing the indicator within a higher molecular weight complex. Preferred support materials have a molecular weight of at least 500, for example at least 1000, 1500 or 2000 or 10,000. The support material should also be soluble in water, and should be inert in the sense that it does not interfere with the sensor itself.

Suitable materials for use as the support material include polymers. Any non-cross-linked, linear polymer which is soluble in the solvent used can be employed. Alternatively, the support material may be a cross linked polymer (e.g. a lightly cross-linked polymer) that is capable of forming a hydrogel in water. For example, the support material may be a hydrogel formed from a cross-linked polymer having a water content of at least 30% such that there is no distinct interface between the polymer and aqueous domains.

Polyacrylamide and polyvinylalcohol are examples of appropriate water-soluble, linear polymers. Preferably, the polymer used has a low polydispersity. More preferably, the polymers are uniform (or monodisperse) polymers. Such polymers are composed of molecules having a uniform molecular mass and constitution. The lower polydispersity leads to an improved sensor modulation. Cross-linked polymers for formation of hydrogels may be formed from the above water-soluble linear polymers cross-linked with ethylene glycol dimethacrylate and/or hydroxylethyldimethacrylate.

In one embodiment, the indicator is bound to a hydrogel having a high water content. In this instance, the indicator system typically comprises an aqueous solution containing the hydrogel. The water content of the hydrogel is so high, preferably at least 30% w/w, that the solution/hydrogel mixture can be considered a mixture of fluids with no distinct solid interfaces between the polymer and aqueous domains. As used herein, a fluid hydrogel is a hydrogel having a water content which is so high (typically at least 30% w/w) that there are no distinct solid interfaces between the polymer and aqueous domains when the hydrogel is placed in water. Such a hydrogel may comprise a lightly cross-linked polymer which may dissolve in the solvent, or which may form a fluid hydrogel with a relatively low water content; alternatively, the hydrogel may comprise a more heavily cross-linked polymer having a higher water content such that it is in the form of a fluid.

In a particularly preferred aspect, the support material is a dendrimer. The nature of the dendrimer for use in the invention is not particularly limited and a number of commercially available dendrimers can be used, for example polyamidoamine (PAMAM), e.g. STARBURST® dendrimers and polypropyleneimine (PPD, e.g. ASTRAMOL® dendrimers. Other types of dendrimers that are envisaged include phenylacetylene dendrimers, Frechet (i.e. poly(benzylether)) dendrimers, hyperbranched dendrimers and polylysine dendrimers. In one aspect of the invention a polyamidoamine (PAMAM) dendrimer is used.

Dendrimers include both metal-cored and organic-cored types, both of which can be employed in the present invention. Organic-cored dendrimers are generally preferred.

The properties of a dendrimer are influenced by its surface groups. In the present invention, the surface groups act as the binding point for attachment to the receptor and the fluorophore. Preferred surface groups therefore include functional groups which can be used in such binding reactions, for example amine groups, ester groups or hydroxyl groups, with amine groups being preferred. The nature of the surface group, however, is not particularly limited. Some conventional surface groups which could be envisaged for use in the present invention include amidoethanol, amidoethylethanolamine, hexylamide, sodium carboxylate, succinamic acid, trimethoxysilyl, tris(hydroxymethyl)amidomethane and carboxymethoxypyrrolidinone, in particular amidoethanol, amidoethylethanolamine and sodium carboxylate.

The number of surface groups on the dendrimer is influenced by the generation of the dendrimer. Preferably, the dendrimer has at least 4, more preferably at least 8 or at least 16 surface groups. Typically, all of the surface groups of the dendrimer will be bound to a receptor or fluorophore moiety. However, where some surface groups of the dendrimer remain unbound to a receptor or fluorophore moiety (or a construct of receptor and fluorophore), the surface groups may be used to impart particular desired properties. For example, surface groups which enhance water-solubility such as hydroxyl, carboxylate, sulphate, phosphonate or polyhydroxyl groups may be present. Sulphate, phosphonate and polyhydroxyl groups are preferred examples of water soluble surface groups.

In one aspect, the dendrimer incorporates at least one surface group which contains a polymerisable group. The polymerisable group may be any group capable of undergoing a polymerisation reaction, but is typically a carbon carbon double bond. Examples of suitable surface groups incorporating polymerisable groups are amido ethanol groups wherein the nitrogen atom is substituted with a group of formula-linker-C═CH₂. The linker group is typically an alkylene, alkylene-arylene, or alkylene-arylene-alkylene group wherein the alkylene is typically a C1 or C2 alkylene group and arylene is typically phenylene. For example, the surface group may comprise an amidoethanol wherein the nitrogen atom is substituted with a —CH₂—Ph—CH═CH₂ group.

The presence of a polymerisable group on the surface of the dendrimer enables the dendrimer to be attached to a polymer by polymerising the dendrimer with one or more monomers or polymers. Thus, the dendrimer can be tethered to, for example, a water soluble polymer in order to enhance water solubility of the dendrimer, or to a hydrogel (i.e. a highly hydrophilic cross-linked polymer matrix, e.g. of polyacrylamide) to assist in containing the dendrimer within the cell.

Preferably the dendrimer is symmetrical, i.e. all of the dendrons are identical.

The dendrimer may have the general formula:

CORE-[A]ₙ

wherein CORE represents the metal or organic (preferably organic) core of the dendrimer and n is typically 4 or more, for example 8 or more, preferably 16 or more. Examples of suitable CORE groups include benzene rings and groups of formula —RN—(CH₂)ₚ—NR— and N—(CH₂)ₚ—N where p is from 2 to 4, e.g. 2 and R is hydrogen or a C1-C4 alkyl group, preferably hydrogen. —HN—(CH₂)₂—NH— and N—(CH₂)₂—N are preferred.

Each group A may be attached either to the CORE or to a further group A, thus forming the typical cascading structure of a dendrimer. In a preferred aspect, 2 or more, for example 4 or more, groups A are attached to the CORE (first generation groups A). The dendrimer is typically symmetrical, i.e. the CORE carries 2 or more, preferably 4 or more, identical dendrons.

Each group A is made up of a basic structure having one or more branching groups. The basic structure typically comprises alkylene or arylene moieties or a combination thereof. Preferably the basic structure is an alkylene moiety. Suitable alkylene moieties are C1-C6 alkylene moieties. Suitable arylene moieties are phenylene moieties. The alkylene and arylene moieties may be unsubstituted or substituted, preferably unsubstituted, and the alkylene moiety may be interrupted or terminated with a functional group selected from —NR′—, —O—, —CO—, —COO—, —CONR′—, —OCO— and —OCONR′, wherein R′ is hydrogen or a C1-C4 alkyl group.

The branching groups are at least trivalent groups which are bonded to the basic structure and have two or more further points of attachment. Preferred branching groups include branched alkyl groups, nitrogen atoms and aryl or heteroaryl groups. Nitrogen atoms are preferred.

The branching groups are typically bonded to (i) the basic structure of the group A and (ii) to two or more further groups A. Where on the surface of the dendrimer, however, the branching group may itself terminate the dendrimer (i.e. the branching group is the surface group), or the branching group may be bonded to two or more surface groups.

Examples of preferred groups A are groups of formula

—(CH₂)_{q}—(FG)ₛ—(CH₂)ᵣ—NH₂

wherein q and r are the same or different and represent an integer of from 1 to 4, preferably 1 or 2, more preferably 2. s is 0 or 1. FG represents a functional group selected from —NR′—, —O—, —CO—, —COO—, —CONR′—, —OCO— and —OCONR′, wherein R′ is hydrogen or a C1-C4 alkyl group. Preferred functional groups are —CONH—, —OCO— and —COO—, preferably —CONH—.

A discussed above, the surface group forms the point of attachment of the dendrimer to the indicator (or separately to the receptor and fluorophore moieties). The surface groups therefore typically include an unsubstituted or substituted alkylene or arylene moiety or a combination thereof, preferably an unsubstituted or substituted alkylene moiety, and at least one functional group which is suitable for bonding to the indicator. The functional group is typically an amine or hydroxyl group, with amine groups being preferred. Particular examples of surface groups are provided above.

Where the dendrimer employed is a metal-cored dendrimer, it may itself have fluorescent properties. In this case, it is envisaged that the dendrimer itself may form the fluorophore moiety. The support-bound indicator in this case simply comprises a receptor moiety bound to the dendrimer.

In a further aspect, the support material is a non-dendritic, non-polymeric macromolecule having high molecular weight (i.e. at least 500, preferably at least 1000, 1500 or 2000 or 10,000). Cyclodextrins, cryptans and crown ethers are examples of such macromolecules. Such macromolecules also provide a uniform environment for the indicator and lead to a more consistent fluorophore response to analyte binding.

The receptor and fluorophore may be bonded to the support material by any appropriate means. Covalent linkages are preferred. Typically, the fluorophore and receptor are linked to form a fluorophore-receptor construct, which is then bound to the support material. Alternatively, the receptor and fluorophore may be separately bound to the support material. The number of receptor-fluorophore construct moieties per support material moiety is typically greater than 1, for example 4 or more, or 8 or more. Where a dendritic support material is used, the surface of the dendrimer may be covered with indicator moieties. This may be achieved by binding an indicator moiety to all (or substantially all) of the surface dendrons.

Where a polymeric support material is used, the receptor-fluorophore construct may be modified to include a double bond and copolymerised with a (meth)acrylate or other appropriate monomer to provide a polymer bound to the indicator. Alternative polymerisation reactions, or simple addition reactions, may also be employed. Wang et al (Wang B., Wang W., Gao S., (2001), Bioorganic Chemistry, 29, 308-320) provides an example of a polymerisation reaction including a monoboronic acid glucose receptor linked to an anthracene fluorophore.

In the case of a dendritic support material, the dendrimer is either reacted separately with the fluorophore and receptor moieties, or more preferably is reacted with a pre-formed receptor-fluorophore construct. Any appropriate binding reaction may be used. An example of a suitable technique is to react a dendrimer having surface amine groups with a fluorophore-receptor construct having a reactive aldehyde group by reductive amination in the presence of a borohydride type reagent. The resulting structure can be purified by ultrafiltration. An example of a dendrimer bound to a boronic acid receptor and an anthracene fluorophore is provided by James et al (Chem. Commum., 1996 p706).

In the case of the dendritic support material having a polymerisable group as a surface group, the dendrimer may undergo a polymerisation reaction with one or more monomers in order to form a dendrimer-polymer construct wherein a polymer is bound to the surface of the dendrimer. Typically, the dendrimer is added at a late stage in the polymerisation reaction so that the dendrimer terminates the polymer chain.

Alternatively, the dendrimer may be reacted with a pre-formed polymer. This can be achieved, for example, by a condensation reaction between a carboxylic acid group on the polymer with a hydroxyl group on the dendrimer, to provide the link through the formed ester.

Examples of monomers and polymers which can be used in these reactions are (meth)acrylate, (meth)acrylamide and vinylpyrrolidone and combinations thereof and their corresponding polymers. Preferred polymers are water soluble polymers. Preferably, the water-solubility of the polymer is such that adequate fluorescent signal is produced when the polymer/indicator is dissolved in water (ideally infinite solubility). Polyacrylamide is particularly preferred since this leads to the formation of a highly water soluble polyacrylamide chain attached to the dendrimer. In one aspect of this embodiment, the polymer (e.g. polyacrylamide) chain bound to the dendritic support material is cross-linked to form a hydrogel. Optionally, the hydrogel has a high water content such that when placed in water there is no distinct interface between the aqueous phase and the polymer phase (as used herein, the hydrogel is in fluid form). In this case, it is typically provided in the form of a mixture with water or an aqueous solution.

Polymerisation from the surface of the dendrimer may be carried out either before or after attachment of the fluorophore and receptor moieties.

In the case of a the receptor and fluorophore being provided to the sensor in aqueous solution, a suitable concentration of receptor-fluorophore construct or support bound construct is 10⁻⁶ to 10⁻³ M . The concentration may be varied dependent on the required sensor properties. The higher the concentration or amount of receptor and fluorophore in the solution, the greater the signal level.

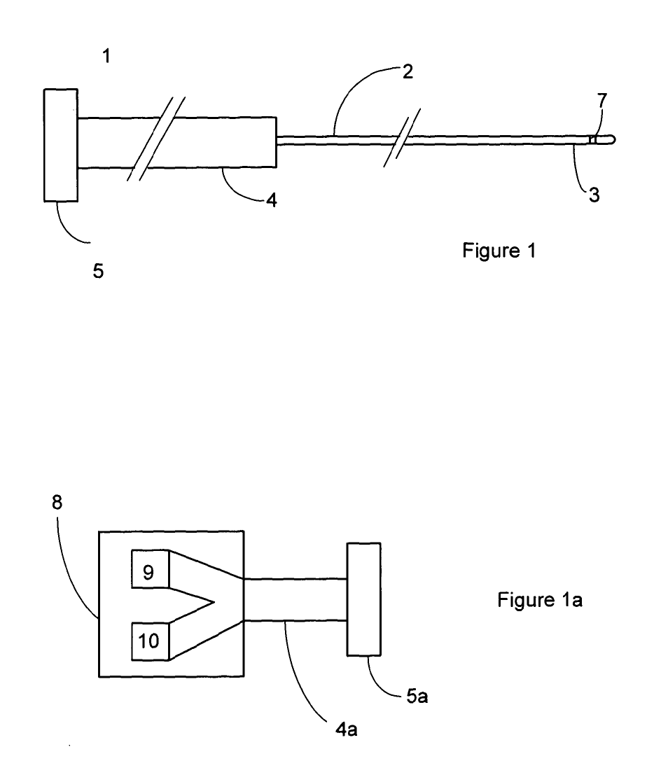

An example of a sensor of the invention is depicted in FIGS. 1 and 1a. The sensor 1 comprises an optical fibre 2 including a sensing region 3 at its distal end. Fibre 2 is adapted for insertion into the blood vessel of a patient, for example through a cannular.

The sensors of the invention are adapted for intravascular use and therefore must be capable of insertion into a blood vessel, typically a vein or artery. Typically, the sensor of the invention is inserted through a cannula, such as a standard 20 gauge cannula. Accordingly, the sensor generally has a maximum diameter of 0.5 mm in the section which is to enter the blood vessel (in FIG. 1 and 1a the sensing region 3 of the fibre has a maximum diameter of 0.5 mm). The length of the sensor is generally at least 5 cm to enable the fibre to pass through the cannula and such that the sensing region is located within the blood vessel and does not remain within the cannula. Typically, the sensor will comprise a fibre which is significantly longer than 5 cm, with only a distal part of the fibre, incorporating the sensing region, entering the blood vessel.

The sensing region 3 contains a cell or chamber 7 in which the indicator system is contained. The optical fibre extends through cable 4 to connector 5 which is adapted to mate with an appropriate monitor 8. The monitor typically includes further optical cable 4a that mates with the connector at 5a and at the other bifurcates to connect to (a) an appropriate source of incident light for the optical sensor 9 and (b) a detector for the return signal 10.

As depicted in FIG. 1, the sensing region 3 incorporates a cell 7 in the form of a chamber within the fibre. The cell may take any form, as long as it enables the indicator system to be contained in the path of the incident light directed by the optical fibre. Thus, the cell may be attached to the distal end of the fibre or may be in the form of a chamber within the fibre having any desired shape. The cell has at least one aperture (not depicted) to allow entry of glucose from the blood stream into the cell.

In one embodiment, the receptor/fluorophore are provided in a hydrogel or other polymeric matrix. Alternatively, they may be provided in aqueous solution. Glucose-permeable membrane is preferably placed across the or each aperture to maintain the indicator system within the cell and allow entry of glucose.

In one embodiment of the invention, the fluorescent signal may be temperature corrected. In this embodiment, a thermocouple (thermistor or other temperature probe) will be place beside the indicator system in or on the distal end of the fibre.

Also provided in the sensor of the invention is a light source 9 for transmitting incident light of appropriate wavelength to the indicator and a detector 10 for detecting a return signal. The light source is preferably an LED but may be an alternative light source such as a laser diode. The light source may be temperature stabilised. The wavelength of the light source will depend on the fluorophore used. The term “light” is not intended to imply any particular restriction on the emission wavelength of the light source, and in particular is not limited to visible light. The light source 9 may include an optical filter to select a wavelength of excitation, but this filtering may be unnecessary if the light source has a sufficiently narrow band or is monochromatic.

Any appropriate detector 10 capable of detecting fluorescence lifetimes may be used. In one aspect the detector 10 is a single photon avalanche diode (SPAD) (a type of photodiode). Suitable SPADs include SensL SPMMicro, Hamamatsu MPPC, Idquantique ID101, and other similar devices. (A single-photon avalanche diode may also be known as a Geiger-mode APD or G-APD; where APD stands for avalanche photodiode.) An optical filter (not shown) may be provided to restrict the wavelengths of light that can reach the detector 10, for instance to block substantially all light except that at the fluorescence wavelength of interest.

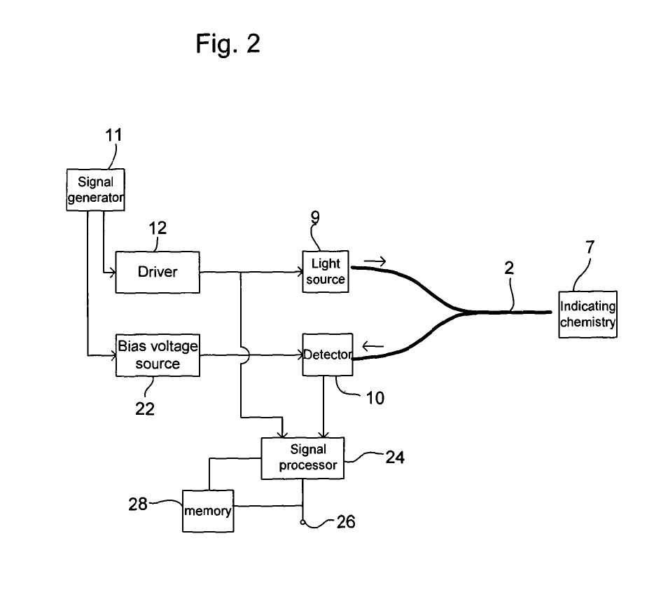

FIG. 2 shows schematically a preferred embodiment of a fluorescence sensor according to the invention which uses a SPAD detector. This embodiment describes the measurement of the lifetime of the fluorophore using frequency domain measurements, but the same apparatus can equally be used for time domain measurements. A signal generator 11 produces a high frequency periodic signal at a first frequency that is passed to a driver 12. The driver 12 may condition the first signal and then uses it to drive modulation of the light source 9.

The driver 12 drives the light source 9 to modulate the intensity (amplitude) of the excitation light. Preferably this is done by the driver 12 electrically modulating the light source to vary the emission intensity. Alternatively, the light source 9 may include a variable optical modulator to change the final output intensity. The shape (waveform) of the modulation of the intensity of the light from the light source 9, controlled by the signal generator 11 and the driver 12, may take various forms depending on the circumstances, including sinusoidal, triangular or pulsed, but the modulation is periodic at the first frequency.

The light output from the light source 9 is transmitted to the indicator system in cell 7 via optical fibre 2. In this embodiment, because the output of the light source 9 is periodically modulated, then the fluorescence light is also modulated in nature at the same fundamental first frequency. However, there is a time delay introduced in the fluorescence emitted light because of the fluorescence behaviour of the fluorophore; this manifests itself as a phase delay between the modulation of the excitation light and the modulation of the fluorescence light.

The emitted fluorescence light is transmitted to a detector 10 via optical fibre 2. In this embodiment, detector 10 is a single photon avalanche diode (SPAD). The single photon avalanche diode detector 10 can be either the kind having a low breakdown voltage (threshold) or a high breakdown voltage. A bias voltage may be applied to the single photon avalanche diode detector by a bias voltage source 22, such that the bias voltage is above the breakdown voltage of the single photon avalanche diode. In this state the detector 10 has very high sensitivity such that receipt of a single photon causes an output current pulse, and thus the total output current is related to the received light intensity, even when the intensity is very low.

The bias voltage source 22 receives a periodic signal at a second frequency from the signal generator 11 such that the bias voltage applied to the single photon avalanche diode detector 10 is modulated at that second frequency. In the preferred embodiment, the single photon avalanche diode detector is a low voltage type and the mean bias voltage is in the region of 25 to 35 Vdc, but may be higher or lower depending on the actual device breakdown voltage, with a modulation depth of typically 3 to 4 V at the second frequency. The waveform of the modulation, like that of the light source, is not limited to any particular form, but is typically sinusoidal. The output of the detector 10 is passed to a signal processor 24. An analogue-to-digital converter (ADC) (not shown) can be provided so that the analogue output signal of the single photon avalanche diode is converted to the digital domain and the signal processor 24 can employ digital signal processing (DSP).

The signal processor 24 can be implemented in dedicated electronic hardware, or in software running on a general purpose processor, or a combination of the two. In a preferred embodiment, a microprocessor (not shown) controls both the signal processor 24 that performs the analysis, and the signal generator 11. Thus the signal processor 24 has information on the light source modulation signal frequency and phase, and the detector bias voltage modulation frequency and phase.

The modulation of the bias voltage modulates the gain of the single photon avalanche diode detector 10. The light source 9, and hence the received fluorescence light are modulated at a first frequency, but the bias voltage of the single photon avalanche diode detector 10 is modulated at a second frequency, different from the first frequency. This enables a heterodyne measurement approach to be used by the signal processor 24 operating on an analysis signal at a frequency equal to the difference between the first frequency and the second frequency. Preferably the first and second frequencies differ by less than 10%, more preferably by less than 1%. The difference in frequency between first and second frequencies depends on the indicator system used but may be, for example 50 kHz.

According to another embodiment, the first and second frequencies can be nominally the same, but a varying phase shift is introduced between the signals (for example by delaying one signal with respect to the other, by a delay that continuously varies). As the phase shift changes each cycle, this is in fact the same as having two different frequencies. Preferably the introduced phase shift is swept rapidly.

From the signal being analysed, and knowing the frequency and phase of both the modulation of the light source 9 and of the modulation of the detector bias voltage, the signal processor 24 can determine the phase delay introduced into the system.

The phase delay intrinsic to the sensor (which can be calculated either without any fluorophore present or with a sample of known fluorescence lifetime (known phase delay)) is deducted, providing a phase shift due purely to the fluorophore in the indicator system. This information can then be converted to a glucose concentration using appropriate calibration data. The required measurement result is then presented at output 26. The output measurement result can be displayed on a display (not shown) and/or can be logged in a memory 28 for later retreival. .

The above-described method essentially uses a single data point to derive the desired fluorescence-related information. However, according to a further preferred embodiment of the invention, a series of measurements are performed, but for each measurement a different phase shift and/or frequency difference is electronically introduced such that the phase angle can be controllably advanced or retarded. The two signal waveforms generated by the signal generator 11 are at the first and second frequencies that are different from each other, such that the relative phase of the signals at these frequencies will vary with time. However, the apparatus is in control, so that, for example, the waveforms at the two frequencies can be synchronised at a particular instant, and then the actual phase shift at any other time can be calculated. In one example, measurements are repeated with shifts in the frequency difference of 10 kHz, 20 kHz and 30 kHz. In addition a specific phase shift can be introduced at the point of synchronisation, so that the waveforms have a known initial phase difference. For each introduced phase angle shift, the modulation depth of the signal being analysed is obtained in order to effectively map out the phase-modulation space. The introduced phase angle may be incremented for example in steps of 5 degrees from zero to 180 degrees. The result is a series of data points that relate the modulation depths to the introduced phase angles. These data points constitute a graph that can be analysed e.g. by curve-fitting and/or comparison with calibration data of modulation depth relative to phase angle either with no sample present or with one or more standard calibration samples present. In general terms, results of measurements using different initial phase differences and/or different frequency differences can be aggregated, thus the overall measurement accuracy can be improved.

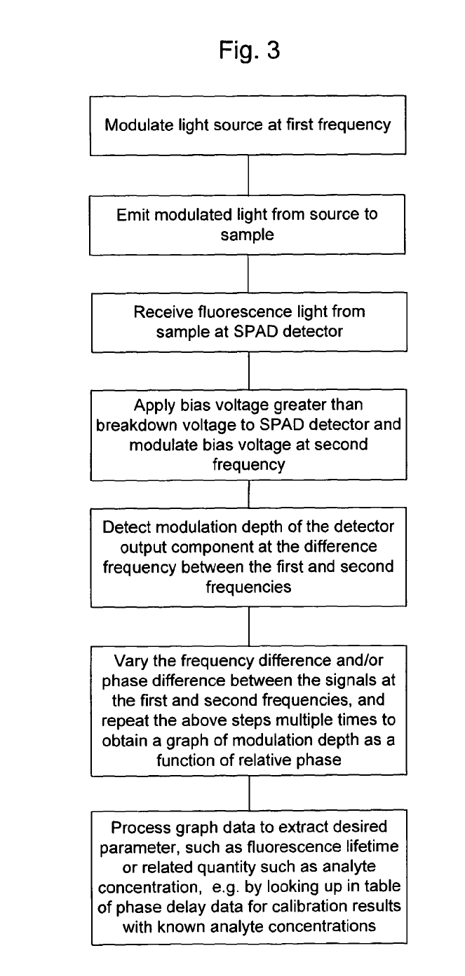

A summary of the method described above is depicted schematically in the flowchart of FIG. 3.

The whole sensor apparatus can be controlled by a microprocessor (not depicted). Although FIG. 2 shows a number of discrete electronic circuit items, at least some of these may be integrated in a single integrated circuit, such as a field-programmable gate array (FPGA) or application-specific integrated circuit (ASIC).

The invention has been described with reference to various specific embodiments and examples, but it should be understood that the invention is not limited to these embodiments and examples.

1. A glucose sensor for intravascular measurement of glucose concentration wherein the sensor is arranged to measure glucose concentration by monitoring the lifetime of the fluorophore, the sensor comprising:
an indicator system comprising a receptor for selectively binding to glucose and a fluorophore associated with said receptor, wherein the fluorophore has a lifetime of less than 100 ns;
a light source;
an optical fibre arranged to direct light from the light source onto the indicator system;
a detector arranged to receive fluorescent light emitted from the indicator system; and
a signal processor arranged to determine information related to a fluorescence lifetime of the fluorophore based on at least the output signal of the detector.

2. A sensor according to claim 1, wherein the detector is a single photon avalanche diode.

3. A sensor according to claim 2, further comprising:
a driver arranged to modulate the light source intensity at a first frequency;
a bias voltage source arranged to apply a bias voltage to the single photon avalanche diode, wherein the bias voltage is modulated at a second frequency, different from the first frequency, and wherein the bias voltage is above the breakdown voltage of the single photon avalanche diode.

4. A sensor according to claim 3, wherein the signal processor operates on a component of the output signal of the single photon avalanche diode at a frequency given by the difference between the first and second frequencies.

5. A sensor according to claim 3, wherein a signal generator is controlled to vary at least one of: the frequency difference between said first and second frequencies; and the phase difference between signals at said first and second frequencies used to modulate the light source and modulate the bias voltage.

6. A sensor according to claim 1, wherein the indicator system comprises a fluorophore-receptor construct which is bound to a hydrogel.

7. A sensor according to claim 6, wherein the hydrogel is a fluid hydrogel having a water content of at least 30% w/w.

8. A sensor according to claim 1, wherein the indicator system is an aqueous solution in which the receptor and fluorophore are dissolved.

9. A sensor according to claim 1, wherein the fluorophore has a lifetime of 30 ns or less.

10. A sensor according to claim 1, wherein the fluorophore is a non-metallic fluorophore.

11. A method of intravascular measurement of glucose concentration comprising
inserting the indicator system of a sensor as defined in claim 1 or 6 to into a vein or artery;
passing incident light from the light source to the indicator system via the optical fibre;
receiving fluorescent light, emitted from the indicator system in response to the light incident on the indicator system from the light source, using the detector and generating an output signal; and
determining information related to the fluorescence lifetime of the fluorophore based on at least the output signal of the detector.

12. A method according to claim 10, wherein the detector is a single photon avalanche diode and the method further comprises the steps of:
modulating the light source intensity at a first frequency; and
applying a bias voltage to the single photon avalanche diode, wherein the bias voltage is modulated at a second frequency, different from the first frequency, and wherein the bias voltage is above the breakdown voltage of the single photon avalanche diode.

13. A method according to claim 12, comprising determining the fluorescence lifetime information based on a component of the output signal of the single photon avalanche diode at a frequency given by the difference between the first and second frequencies.

14. A method according to claim 12, further comprising at least one of: varying the frequency difference between the first and second frequencies; and controlling the phase difference between signals at said first and second frequencies used to modulate the light source and modulate the bias voltage.
